(19) United States
(12) Patent Application Publication
(10) Pub. No.: US 2020/0297307 A1
(43) Pub. Date: Sep. 24, 2020

(54) Catheter with Seal Layer

(71) Applicant: Avent, Inc., Alpharetta, GA (US)

(72) Inventors: Steve S. Khalaj, Laguna Hills, CA (US); Shirzad Shahriari, Irvine, CA (US)

(21) Appl. No.: 16/358,861

(22) Filed: Mar. 20, 2019

(51) Int. Cl. A61B 8/12; A61B 5/00; A61B 8/00

(52) U.S. Cl. CPC A61B 8/12; A61B 2090/378; A61B 8/445; A61B 5/0084

(57) ABSTRACT

A catheter assembly for locating an object of interest (e.g., an anatomical region such as a muscle, nerve bundle, etc.) is provided. The catheter assembly includes a catheter having a catheter body defining a proximal end and a distal end; a transducer located at the distal end of the catheter; one or more transducer wires extending from a proximal end of the transducer towards the proximal end of the catheter; and a length of tubing surrounding the catheter, the transducer, and the one or more transducer wires, wherein the length of tubing has a proximal portion and a distal portion, wherein the proximal portion surrounds the one or more transducer wires and the distal portion surrounds the transducer, wherein the proximal portion includes a conductive filler.

### FIELD OF THE INVENTION

The present invention relates generally to medical devices used in conjunction with ultrasound imaging systems, and more particularly to devices used in medical procedures such as nerve blocks, biopsies, or any other procedure where access is provided via a catheter.

### BACKGROUND OF THE INVENTION

Detection of anatomical objects using medical imaging is an essential step for many medical procedures, such as regional anesthesia nerve blocks, and is becoming the standard in clinical practice to support diagnosis, patient stratification, therapy planning, intervention, and/or follow-up. Various systems based on traditional approaches exist for anatomical detection and tracking during medical procedures, such as computed tomography (CT), magnetic resonance (MR), ultrasound, and fluoroscopic imaging systems.

For example, ultrasound imaging systems utilize sound waves with frequencies higher than the upper audible limit of human hearing. Further, ultrasound imaging systems are widely used in medicine to perform both diagnosis and therapeutic procedures. In such procedures, sonographers perform scans of a patient using a hand-held probe or transducer that is placed directly on and moved over the patient.

One problem during procedures that utilize ultrasound imaging, such as regional anesthesia nerve block procedures or biopsy procedures, is the accurate placement of a needle in order to deliver the nerve block (e.g., via the delivery of an anesthetic through the needle, via the insertion of a catheter to deliver the nerve block via RF energy, or via the combination of the delivery of an anesthetic and RF energy) to the desired nerve bundle or to biopsy the correct tissue sample. Thus, the placement of an ultrasonic transducer at the tip of the needle can be used in conjunction with an ultrasound imaging system to visualize the location of the tip of the needle to assist a medical professional in accurately positioning the tip of the needle.

However, often, a catheter-based infusion system is utilized to block the nerve bundles at the incision after surgery to provide a continuous, low flow rate of the anesthetic over a period of time (e.g., 2-5 days following surgery) for post-operative pain management. Often, it is desirable to use a catheter-based system that has a rounded or blunt distal end or tip instead of a needle or other introducer that has a pointed distal tip, as such a configuration may allow the catheter assembly to be placed in, or remain at, a target area while causing less damage to nerves or tissues than a sharp or pointed introducer, tunneler, or needle based infusion system. Therefore, as the needle, when used, is generally removed after placement of the catheter, it can be difficult for medical professionals to easily place, or confirm the continued placement of the catheter after removal of the needle containing the transducer.

Further, the ultrasonic transducer is typically connected to an external power supply via a large coaxial cable in order to minimize the signal noise associated with the electrical connection between the ultrasonic transducer and the external power supply. While minimizing the signal noise is important, the size of the typical coaxial cable required to sufficiently minimize the signal noise can be problematic when it is desired to advance the transducer assembly to a nerve bundle or tissue or to remain connected to a patient for a period of time. For instance, the increased diameter of the transducer assembly due to the presence of a large coaxial cable can make it difficult for the transducer assembly to reach a target nerve bundle or tissue located beneath several layers of skin, muscle, etc, and also becomes problematic as the large size may increase the likelihood of contact or pulling by the patient, increasing the changes of movement from the target.

As such, a need exists for a catheter assembly that includes an ultrasonic transducer to enable periodic monitoring of the placement of the catheter assembly. Additionally, a need exists for a means of electrically connecting an ultrasonic transducer to an external power supply where the connection has a reduced size while still effectively minimizing the signal noise through the wire.

### SUMMARY

Objects and advantages of the invention will be set forth in part in the following description, or may be obvious from the description, or may be learned through practice of the invention. The invention will be described in greater detail below by reference to embodiments thereof illustrated in the figures.

According to one embodiment of the present invention, a catheter assembly is provided. The catheter assembly includes a catheter having a catheter body defining a proximal end and a distal end, and a tip located at the distal end of the catheter, the tip being capable of being inserted into a body of a mammal; a transducer located at the distal end of the catheter proximal to the tip; one or more transducer wires extending from a proximal end of the transducer towards the proximal end of the catheter; and a length of tubing surrounding the catheter, the transducer, and the one or more transducer wires, wherein the length of tubing has a proximal portion and a distal portion, wherein the proximal portion surrounds the one or more transducer wires and the distal portion surrounds the transducer.

In one particular embodiment, the transducer can be disposed on an outer surface of the catheter. Further, the length of tubing can hold the transducer in place against the outer surface of the catheter.

In another embodiment, the length of tubing can include polytetrafluoroethylene, polyethylene, polyurethane, silicone, or a combination thereof.

In still another embodiment, the length of tubing can be shrinkable. For instance, the length of tubing can be heat shrinkable.

In another embodiment, the proximal portion of the tubing can include a conductive filler.

In one more embodiment, the conductive filler can include a metal, a conductive polymer, carbon, or a combination thereof. For example, in some embodiments, the conductive filler can include nickel, copper, silver, gold, platinum, or a combination thereof. In other embodiments, the conductive filler can include polypyrrole, poly(3,4-ethylenedioxythiophene), polythiophene, polyaniline, poly-p-phenylene-sulphide, polyacetylene, polyisoprene, polybutadiene, or a combination thereof.

In one particular embodiment, the conductive filler can be present in the proximal portion of the length of tubing in an amount ranging from about 0.05 wt. % to about 10 wt. % based on the total weight of the proximal portion of the length of tubing.

In one embodiment, a human tissue impedance matching material can be disposed between the transducer and the distal portion of the tubing.

In still another embodiment, the one or more transducer wires can have a diameter ranging from about 15 micrometers to about 300 micrometers.

In one more embodiment, the catheter assembly includes a human tissue impedance matching material disposed between the transducer and the distal portion of the tubing.

In one particular embodiment, the distal end of the catheter includes at least one aperture.

In still another embodiment, the transducer can be electrically connected to an external power supply via the one or more transducer wires.

In yet another embodiment, the transducer can transmit and receive signals for detection by an ultrasound imaging system, wherein the transducer enhances the visibility of the distal end of the catheter on an ultrasound image.

In a further embodiment, the catheter assembly may include an article configured to be received in the lumen of the catheter, which, in one embodiment, the article may be a needle, stylet, probe, or guidewire.

Additionally or alternatively, the catheter assembly is configured to be received in the lumen of an article, and, in one embodiment, the catheter is disposed within the lumen of an article. In a further embodiment, the article within which the catheter assembly is disposed is a needle, stylet, probe, or guidewire.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention to one skilled in the art, including the best mode thereof, is set forth more particularly in the remainder of the specification, including reference to the accompanying figures, in which:

FIG. 1 is a perspective view of one embodiment of the catheter assembly of the present invention when used in conjunction with an ultrasound imaging system;

FIG. 2 illustrates a block diagram one of embodiment of a controller of an ultrasound imaging system with which the catheter assembly of the present invention is used;

FIG. 3 illustrates one embodiment of the catheter assembly of the present invention;

FIG. 4 is illustrates a side view of a portion of the catheter assembly of FIG. 3 including the catheter, transducer, one or more transducer wires, and a length of tubing which shields the one or more transducer wires connecting the transducer to a power supply from signal noise;

FIG. 5 illustrates one embodiment of the catheter assembly of the present invention;

FIG. 6 illustrates a side view of a portion of the catheter assembly of FIG. 5 including the catheter, transducer, one or more transducer wires, and a length of tubing which shields the one or more transducer wires connecting the transducer to a power supply from signal noise;

FIG. 7 illustrates a schematic diagram of one embodiment of a catheter assembly according to the present disclosure;

FIG. 8 illustrates a cross-sectional view of one embodiment of a needle of the catheter assembly of FIG. 7;

FIG. 9 illustrates a cross-sectional view of one embodiment of a catheter of the catheter assembly according to the present disclosure; and

FIG. 10 illustrates a cross-sectional view of another embodiment of a catheter assembly according to the present disclosure;

Repeat use of reference characters in the present specification and drawings is intended to represent the same or analogous features or elements of the present invention.

### DETAILED DESCRIPTION

Reference will now be made in detail to embodiments of the present invention, examples of which are illustrated in the figures. Each embodiment is provided by way of explanation of the invention and is not meant as a limitation of the invention. For example, features illustrated or described as part of one embodiment may be used with another embodiment to yield still a further embodiment. It is intended that the present invention include these and other modifications and variations coming within the scope and spirit of the invention.

Generally speaking, the present invention is directed to a catheter assembly for locating an object of interest (e.g., an anatomical region such as a muscle, nerve bundle, etc.) or maintaining and/or confirming that the catheter assembly remains at an object of interest for a period of time. For instance, the catheter assembly may generally include a transducer. Thus, the catheter assembly may be initially located at an object of interest, and may also be capable of having the location confirmed, or relocated, after a period of time. The catheter assembly includes a catheter having a catheter body defining a proximal end and a distal end, where the distal end is adjacent to a tip, where the tip may be formed by a distal end of the catheter, or another article, such as a needle, stylet, probe, or guidewire, for example. Regardless of the article that forms the tip, the tip may be shaped and sized so as to be inserted into a body of a mammal. Additionally, the catheter assembly includes a transducer located at the distal end of the catheter and one or more transducer wires extending from a proximal end of the catheter towards the proximal end of the catheter, and a length of tubing surrounding the catheter, the transducer, and one or more transducer wires. The length of tubing has a proximal portion and a distal portion, where the proximal portion surrounds the one or more transducer wires and the distal portion surrounds the transducer, wherein the proximal portion includes a conductive filler.

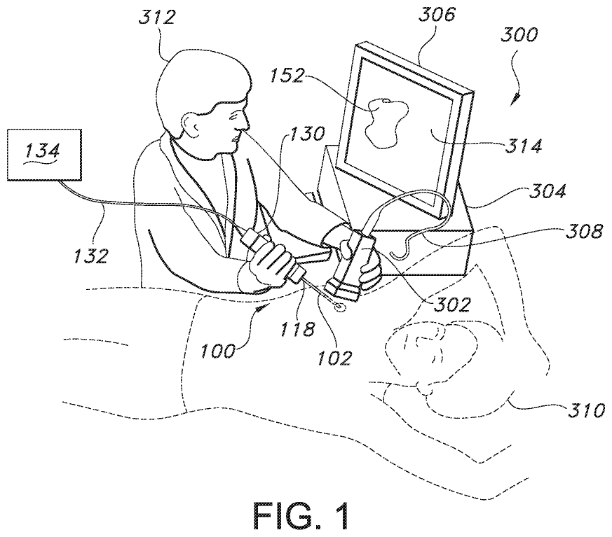

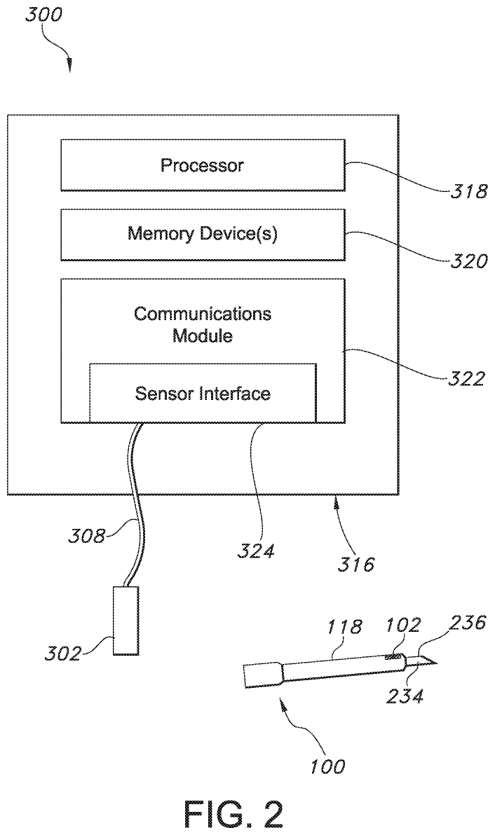

Referring now to the drawings, FIGS. 1 and 2 illustrate a medical imaging system 300 (e.g., an ultrasound imaging system) for use in conjunction with the catheter assembly 100 of the present invention for scanning, identifying, and navigating toward anatomical objects 152 of a patient 310 (e.g., a mammal). As used herein, the anatomical object 152 and surrounding tissue described herein may include any anatomical structure(s) and/or surrounding tissue(s) of a patient 310. For example, in one embodiment, the anatomical object(s) 152 may be a nerve bundle, a muscle, or any other anatomical object that can be visualized in a medical procedure (e.g., nerve block, biopsy, medicament delivery, etc.) performed by a medical professional 312. More specifically, as shown, the medical imaging system 300 may correspond to an ultrasound imaging system or any other suitable imaging system that can benefit from the present technology. Thus, the medical imaging system 300 may generally include a controller 316 having one or more processor(s) 318 and associated memory device(s) 320 configured to perform a variety of computer-implemented functions (e.g., performing the methods and the like and storing relevant data as disclosed herein), as well as a user display 306 configured to display an image 314 of an anatomical object 152 to an operator or medical professional 312. In addition, the medical imaging system 300 may include a user interface 304, such as a computer and/or keyboard, configured to assist the medical professional 312 in generating and/or manipulating the user display 306.

Additionally, as shown in FIG. 2, the controller 316 may also include a communications module 322 to facilitate communications between the processor(s) 318 and the various components of the medical imaging system 300 (e.g., any of the components of FIG. 1). Further, the communications module 322 may include a sensor interface 324 (e.g., one or more analog-to-digital converters) to permit signals transmitted from one or more probes (e.g., the ultrasound imaging probe 302, the catheter assembly 100, or both) to be converted into signals that can be understood and processed by the processor(s) 318. It should be appreciated that the ultrasound imaging probe 302, the catheter assembly 100, or both may be communicatively coupled to the communications module 322 using any suitable means. For example, as shown in FIG. 2, the ultrasound imaging probe 302 may be coupled to the sensor interface 324 via a wired connection 308. However, in other embodiments, the ultrasound imaging probe 302 and/or the catheter assembly 100 may be coupled to the sensor interface 324 via a wireless connection, such as by using any suitable wireless communications protocol known in the art. For example, as shown in FIG. 2, the catheter assembly 100 may be coupled to the sensor interface 324 wirelessly. As such, the processor(s) 318 may be configured to receive one or more signals from ultrasound imaging probe 302 and/or the catheter assembly 100.

As used herein, the term “processor” refers not only to integrated circuits referred to in the art as being included in a computer, but also refers to a controller, a microcontroller, a microcomputer, a programmable logic controller (PLC), an application specific integrated circuit, a field-programmable gate array (FPGA), and other programmable circuits. The processor(s) 318 is also configured to compute advanced control algorithms and communicate to a variety of Ethernet or serial-based protocols (Modbus, OPC, CAN, etc.). Furthermore, in certain embodiments, the processor(s) 318 may communicate with a server through the Internet for cloud computing in order to reduce the computation time and burden on the local device. Additionally, the memory device(s) 320 may generally comprise memory element(s) including, but not limited to, computer readable medium (e.g., random access memory (RAM)), computer readable non-volatile medium (e.g., a flash memory), a floppy disk, a compact disc-read only memory (CD-ROM), a magneto-optical disk (MOD), a digital versatile disc (DVD) and/or other suitable memory elements. Such memory device(s) 320 may generally be configured to store suitable computer-readable instructions that, when implemented by the processor(s) 318, configure the processor(s) 318 to perform the various functions as described herein.

Referring now to FIGS. 1 and 3-6, various embodiments of a catheter assembly 100 that may be visualized via the medical imaging system 300 are illustrated. More specifically, as shown, the catheter assembly 100 can include a catheter 118 having a body 128 having an outer diameter 124 ranging from about 1 millimeters to about 12 millimeters, such as from about 2 millimeters to about 11 millimeters, such as from about 3 millimeters to about 10 millimeters, such as from about 5 millimeters to about 9 millimeters, or any range therebetween, and defined by a distal end 120 and a proximal end 122. A transducer 102 (e.g., an ultrasound transducer) can be disposed on an outer surface 146 of the catheter 118.

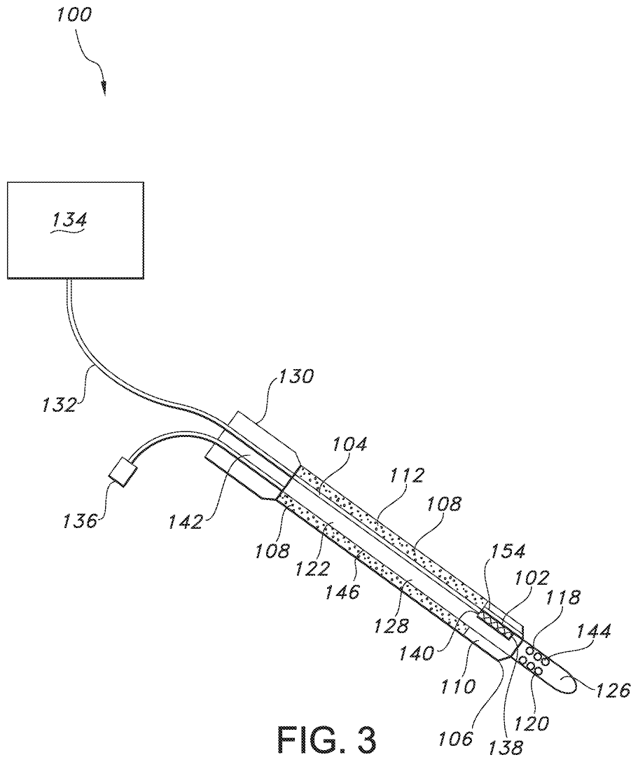

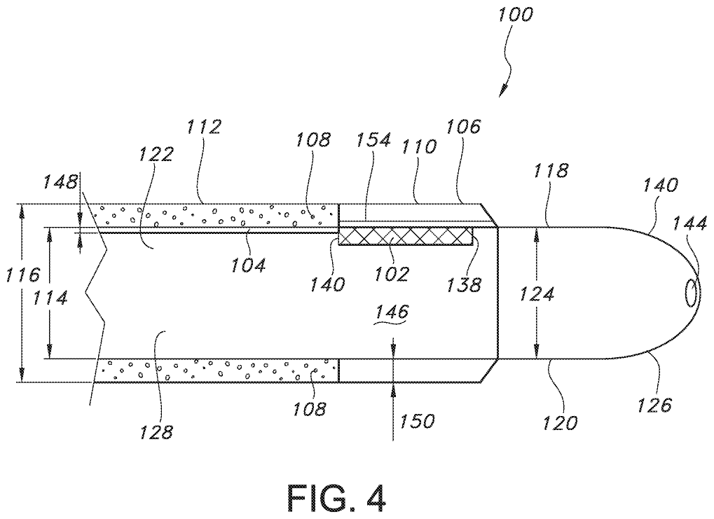

Further, the distal end 120 of the catheter 118 can, in one embodiment, have a tip 126 that can be used for the delivery of energy or treatments suitable for creating a nerve block, such as for the treatment of pain, or may instead utilize openings 144 in the distal end 120 for the delivery of energy or treatments. For instance, the tip 126 and/or distal end 120 can include an opening 144 or openings 144 for delivery of a fluid and/or medicament, such as for delivery of an anesthetic nerve block, or can include a cutting tool (not shown) for taking a biopsy a tissue sample. For instance, in the embodiment shown in FIG. 3, the distal end may include one or more apertures 144 in the distal end 120, for the delivery of fluid and/or medicament. Whereas, in a further embodiment, such as shown in FIG. 4, the aperture 144 or apertures 144 may be located in the tip 126 of the catheter 118 and/or article. While there may be more or less apertures, the catheter body may generally contain at least 1 aperture, such as at least about 2, such as at least about 3, such as at least about 4, such as at least about 5, such as at least about 7 such as at least about 10 apertures. The catheter body may also comprise less than about 20, such as less than about 18, such as less than about 15, such as less than about 13 apertures.

The apertures may be spaced apart radially, grouped in a similar radial plane, axially spaced apart, or both radially and axially spaced apart along the catheter body. In one embodiment, the apertures may be generally located adjacent to the distal end, such as within about 50 millimeters (mm) from the distal end, such as within about 45 mm, such as within about 40 mm, such as within about 35 mm, such as within about 30 mm, such as within about 25 mm, such as within about 20 mm, such as within about 15 mm, such as within about 10 mm of the distal end. In another embodiment, the apertures may be evenly or randomly spaced radially, axially, or a combination of radially and axially along the entirety of the length of the catheter body

The apertures may have a diameter such as about 1 millimeter or less, such as about 900 micrometers or less, such as about 800 micrometers or less, such as about 700 micrometers or less, such as about 600 micrometers or less, such as about 500 micrometers or less, such as about 400 micrometers or less, such as about 300 micrometers or less, such as about 100 micrometers or greater, such as about 200 micrometers or greater, such as about 300 micrometers or greater, such as about 400 micrometers or greater, such as about 500 micrometers or greater, such as about 600 micrometers or greater in diameter. In a further embodiment, some of the apertures may have a fairly large diameter and some of the apertures may have a fairly small diameter. In an embodiment with apertures that have a mixture of diameters, the ratio of the diameter types may be fairly even, or in an alternate embodiment there may be more large diameter apertures, such as twice as many large, such as three times as many large, such as five times as many large diameter apertures. Alternatively, there may be more small diameter apertures such as twice as many small, such as three times as many small, such as five times as many small diameter apertures.

The arrangement of the apertures, number of apertures, and diameter of the apertures may be selected based upon the target area, the type of drug or anesthesia to be delivered, or the desired rate of delivery, to name a few considerations. For example, a larger number of apertures or apertures with a larger diameter, or both, may be selected when a greater rate of delivery is desired. Alternatively, arrangements of apertures may be selected based upon the location and orientation of the target area. For example, if a small target area is desired, all of the apertures may be located fairly close together either radially or axially. In an additional embodiment for example, if a target area may only be located on one side of a catheter, then the apertures may mainly be located on a single side of the catheter body such that the apertures are all located on generally the same side, area, or radius. Alternatively, if a more central location is chosen for the catheter, the apertures may extend radially around the catheter body such that an aperture is located on several different radii around the catheter body.

In one embodiment, the rate of delivery of a drug or anesthetic using a catheter assembly according to the present disclosure may be at least about 0.01 mL/hr, such as about 0.05 milliliters(mL)/hr or greater, such as about 0.1 mL/hr or greater, such as about 0.5 mL/hr or greater, such as about 1 mL/hr or greater, such as about 3 mL/hr or greater, such as about 5 mL/hr or greater, such as about 7.5 mL/hr or greater, such as about 10 mL/hr or greater, such as about 12.5 mL/hr or greater, such as about 15 mL/hr or greater, such as about 17.5 mL/hr or greater, such as about 20 mL/hr or greater, such as about 22.5 mL/hr or greater, such as about 25 mL/hr or greater, such as about 27.5 mL/hr or greater, such as about 30 mL/hr or greater, such as about 35 mL/hr or greater, such as about 40 mL/hr or less, such as about 37.5 mL/hr or less, such as about 35 mL/hr or less, such as about 32.5 mL/hr or less, such as about 30 mL/hr or less, such as about 27.5 mL/hr or less, such as about 25 mL/hr or less, such as about 22.5 mL/hr or less, such as about 20 mL/hr or less, such as about 17.5 mL/hr or less, such as about 15 mL/hr or less, such as about 12.5 mL/hr or less, such as a rate of delivery of about 10 mL/hr or less.

Further, a handle 130 can be coupled to the proximal end 122 of the catheter 118 to assist the medical professional 212 in accurately positioning the catheter 118 within the patient 210. As shown in FIGS. 1 and 3, the handle 130 can be connected via an electrical connection 132 to a power supply 134. The power supply 134 can provide energy to the transducer 102 on the catheter 118. Further, the transducer 102 can enhance the visualization of the catheter assembly 100 (e.g., the distal end 120 of the catheter 118 or the tip 126 of the catheter 118 or article) when used in conjunction with the medical imaging system 200. The transducer 102 can be any piezoelectric element that can transmit ultrasound signals as known in the art. In one particular embodiment, the transducer can be a ceramic piezoelectric transducer. In another embodiment, the transducer can be a capacitive micromachined ultrasonic transducer. It is also to be understood that in addition to transmitting ultrasound signals, the transducer 102 can also receive ultrasound signals, such as from the medical imaging system 300. As shown, the transducer 102 can have a distal end 138 and a proximal end 140, where one or more transducer wires 104, which can connect the transducer 102 to a power supply and which can be used for transmitting and receiving signals, extends from the proximal end 140 of the transducer 102 towards the proximal end 122 of the catheter 118 in order to connect the transducer 102 to the power supply 134. The one or more transducer wires 104, which can extend along the outer surface 146 of the catheter 118, can be formed from any conductive material such as nickel, copper, silver, gold, platinum, or a combination thereof. Further, the one or more transducer wires 104 of the present invention can be non-coaxial in order to decrease the size of the catheter assembly 100, which enables the use of the catheter assembly 100 of the present invention in medical procedures requiring the catheter assembly 100 to have a reduced footprint or diameter. For instance, the one or more transducer wires 104 can have a diameter 148 ranging from about 15 micrometers to about 300 micrometers, such as from about 20 micrometers to about 275 micrometers, such as from about 25 micrometers to about 250 micrometers. The ability of the catheter assembly 100 of the present invention to use one or more transducer wires 104 having such a reduced diameter compared to conventional transducer wires where a larger, more cumbersome coaxial cable is required to shield the one or more transducer wires from electrical signal noise is facilitated by the use of a length of tubing 106 surrounding the catheter 118 and the transducer 102, as discussed in more detail below.

Referring particularly to FIGS. 3-4, the catheter assembly 100 including the catheter 118, the transducer 102, and the one or more transducer wires 104 can also include a length of tubing 106 that surrounds the catheter 118, the transducer 102, and the one or more transducer wires 104, which can be non-coaxial as discussed above. Specifically, the length of tubing 106 can include a distal portion 110 and a proximal portion 112. The distal portion 110 can extend along the body 128 towards the tip 126 of the catheter 118 such that the tip 126 or some or all of the distal portion 110 of the catheter 118 is exposed and free of the length of tubing 106.

As shown, the length of tubing 106 can hold the transducer 102 and one or more transducer wires 104 in place against an outer surface 146 of the catheter 118. As such, the length of tubing 106 can have an inner diameter 114 ranging from about 1 millimeters to about 12 millimeters, such as from about 2 millimeters to about 11 millimeters, such as from about 3 millimeters to about 10 millimeters, such as from about 5 millimeters to about 9 millimeters, or any range therebetween; and an outer diameter 116 ranging from about 1.01 millimeters to about 12.1 millimeters, such as from about 2.01 millimeters to about 11.1 millimeters, such as from about 3.01 millimeters to about 10.1 millimeters, such as from about 5.01 millimeters to about 9.1 millimeters, or any range therebetween.

Thus, the length of tubing 106 can have a wall thickness 150 ranging from about 10 micrometers to about 101 micrometers, such as from about 15 micrometers to about 91 micrometers, such as from about 20 micrometers to about 81 micrometers. The proximal portion 112 of the length of tubing 106 can surround the one or more transducer wires 104 and the distal portion 110 of the length of tubing 106 can surround the transducer 102. Further, the proximal portion 112 of the length of tubing 106 can include a conductive filler 108 (e.g., conductive particles, powder, flakes, etc.) that helps shield the one or more transducer wires 104 from electrical signal noise that may hinder the effectiveness of the transducer 102 in enhancing the visibility of the distal end 120 of the catheter 118. Meanwhile, the distal portion 110 of the length of tubing 106 which surrounds the transducer 102 is free of the conductive filler 108 so as to not distort the signals that may be transmitted and received by the transducer 102, such as when used the medical imaging system 100 to visualize the distal end 120 of the catheter 118.

The length of tubing 106 can include any suitable material that is utilized in medical tubing, such as polytetrafluoroethylene, polyethylene, polyurethane, silicone, or a combination thereof. Further, the length of tubing 106 can be shrinkable, where the length of tubing can secure the transducer 102 and the one or more transducer wires 104 against the outer surface 146 of the catheter 118 when the length of tubing 106 is shrunk around the body 128 of the catheter 118. In one particular embodiment, the length of tubing 106 can be heat shrinkable.

In addition, any suitable conductive filler 108 can be used, such as a metal, a conductive polymer, carbon, or a combination thereof. For instance, the conductive filler 108 can include nickel, copper, silver, gold, platinum, or a combination thereof. In other embodiments, the conductive filler 108 can include polypyrrole, poly(3,4-ethylenedioxythiophene), polythiophene, polyaniline, poly-p-phenylene-sulphide, polyacetylene, polyisoprene, polybutadiene, or a combination thereof. Moreover, regardless of the particular conductive filler 108 utilized, the conductive filler 108 can be present in the proximal portion 112 of the length of tubing 106 in an amount ranging from about 0.05 wt. % to about 10 wt. %, such as from about 0.1 wt. % to about 7.5 wt. %, such as from about 0.5 wt. % to about 5 wt. % based on the total weight of the proximal portion 112 of the length of tubing 106.

Referring now to FIG. 3, the catheter assembly 100 as described above can also include an injection port 136 coupled to the handle 130 whereby fluid or medicament can be injected into a proximal end 122 of the catheter 118. In addition, the catheter assembly 100 can also include an article insertion port 142 located at the handle 130 whereby an article (not shown), such as needle, stylet, probe, or guidewire, for example, can be inserted into the catheter 118 as needed depending on the medical procedure being visualized. In such an embodiment, an article configured to be contained in the lumen 128 of the catheter 118 may be contained in the lumen 128 such that a tip of the article extends through the distal end 120 of the catheter 118, and forms the tip 126. Of course, as discussed in greater detail below, the catheter assembly 100 may also be configured to be contained in the lumen of an article instead of as an over-the-needle catheter.

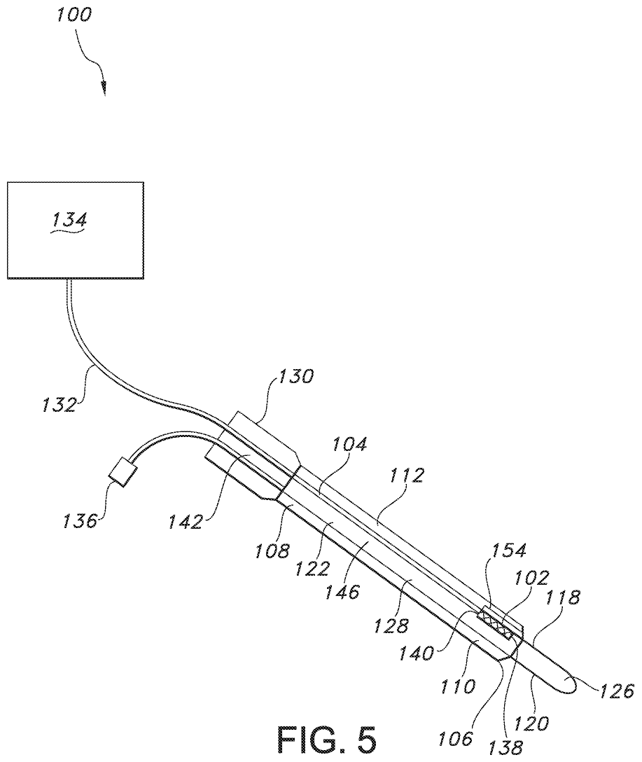

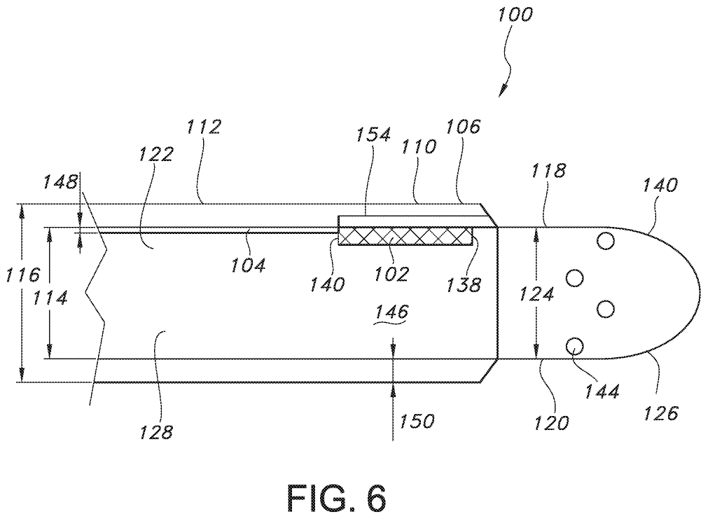

In another embodiment, and referring specifically to FIGS. 5-6, the catheter assembly 100 including the catheter 118, the transducer 102, and the one or more transducer wires 104 can include a length of tubing 106 that surrounds the catheter 118, the transducer 102, and the one or more transducer wires 104, which can be non-coaxial as discussed above. Specifically, the length of tubing 106 can include a distal portion 110 and a proximal portion 112. The distal portion 110 can extend along the body 128 towards the tip 126 of the catheter 118 such that the tip 126 of the catheter 118 is exposed and free of the length of tubing 106. As shown, the length of tubing 106 can hold the transducer 102 and one or more transducer wires 104 in place against an outer surface 146 of the catheter 118.

As such, the length of tubing 106 can have an inner diameter 114 ranging from about 1 millimeters to about 12 millimeters, such as from about 2 millimeters to about 11 millimeters, such as from about 3 millimeters to about 10 millimeters, such as from about 5 millimeters to about 9 millimeters, or any range therebetween; and an outer diameter 116 ranging from about 1.01 millimeters to about 12.1 millimeters, such as from about 2.01 millimeters to about 11.1 millimeters, such as from about 3.01 millimeters to about 10.1 millimeters, such as from about 5.01 millimeters to about 9.1 millimeters, or any range therebetween.

Thus, the length of tubing 106 can have a wall thickness 150 ranging from about 10 micrometers to about 101 micrometers, such as from about 15 micrometers to about 91 micrometers, such as from about 20 micrometers to about 81 micrometers. The proximal portion 112 of the length of tubing 106 can surround the one or more transducer wires 104 and the distal portion 110 of the length of tubing 106 can surround the transducer 102. Further, as shown in FIGS. 5-6, it is to be understood that like the distal portion 110, the proximal portion 112 of the length of tubing 106 can be free of the conductive filler 108 described above with respect to FIGS. 3-4 so as to not distort the signals that may be transmitted and received by the transducer 102, such as when used the medical imaging system 100 to visualize the distal end 120 of the catheter 118.

The length of tubing 106 can include any suitable material that is utilized in medical tubing, such as polytetrafluoroethylene, polyethylene, polyurethane, silicone, or a combination thereof. Further, the length of tubing 106 can be shrinkable, where the length of tubing can secure the transducer 102 and the one or more transducer wires 104 against the outer surface 146 of the catheter 118 when the length of tubing 106 is shrunk around the body 128 of the catheter 118. In one particular embodiment, the length of tubing 106 can be heat shrinkable.

Referring now to FIG. 5, the catheter assembly 100 as described above can also include an injection port 136 to the handle 130 whereby fluid or medicament can be injected into a proximal end 122 of the catheter 118. In addition, the catheter assembly 100 can also include an article insertion port 142 located at the handle 130 whereby an article (not shown), such as needle, stylet, probe, or guidewire, for example, can be inserted into the catheter 118 as needed depending on the medical procedure being visualized. In such an embodiment, an article configured to be contained in the lumen 128 of the catheter 118 may be contained in the lumen 128 such that a tip of the article extends through the distal end 120 of the catheter 118, and forms the tip 126. Of course, as discussed in greater detail below, the catheter assembly 100 may also be configured to be contained in the lumen of an article instead of as an over-the-needle catheter.

Turning to FIGS. 3-6, although not required, in some embodiments, the catheter assembly 100 can include an impedance matching material 154 disposed between the transducer 102 and the distal portion 110 of the tubing 106. The impedance matching material 154 can eliminate any air between the transducer 102 and the distal portion 110 of the tubing 106, where air could attenuate or otherwise negatively impact the signals being transmitted and received by the transducer 102 via the one or more transducer wires 104. In addition, the impedance matching material 154 can have an acoustic impedance that is similar to the impedance of human tissue (e.g., skin), such as an acoustic impedance that is +/−20%, such as +/−15%, such as +/−10%, such as +/−5% of the impedance of human tissue. By having a similar acoustic impedance as human tissue, the impedance matching layer 154 can reduce the possibility of any reflection of the signals being transmitted and received by the transducer 102 via the one or more transducer wires 104. For instance, in one embodiment, the impedance matching layer can be silicone, polyimide, or any other polymer having an acoustic impedance similar to that of human tissue. In addition, the impedance matching material 154 can be applied to the catheter assembly 100 by disposing, such as by coating, dabbing, painting, brushing, etc., a thin layer of the impedance matching material 154 on a surface of the transducer 102 and the distal end 120 of the catheter 118 around which the tubing 106 will be disposed. Further, the impedance matching material 154 and the tubing 106 can be applied around the catheter 118 in a vacuum chamber to ensure that any air is eliminated between the catheter 118 and the tubing 106.

In FIGS. 2-6, the embodiments of the catheter assembly have been described as either a catheter alone, or as an “over the needle” (“OTN”) catheter, where it is noted that the “needle” may be a needle, stylet, probe, or guidewire, for example. For instance, referring back to FIG. 2, a needle 234 may be, and/or may be configured to be located in the lumen (shown more clearly in FIGS. 3-6) of a catheter 118. In such an embodiment, the needle 234 may have a tip 236 that extends through an opening or aperture 144 at a distal end 120 of the catheter 118. As shown, the catheter 118 has a transducer 102 located adjacent to the distal end 120, although, while not shown, it should also be understood that the needle 234 may also contain a transducer, in an embodiment where a needle is used. However, as discussed above, the needle 234 is optional, and in one embodiment, no needle or other article is located in the lumen of the catheter 218, or further yet, the needle may be replaced with a guidewire, stylet, or other article.

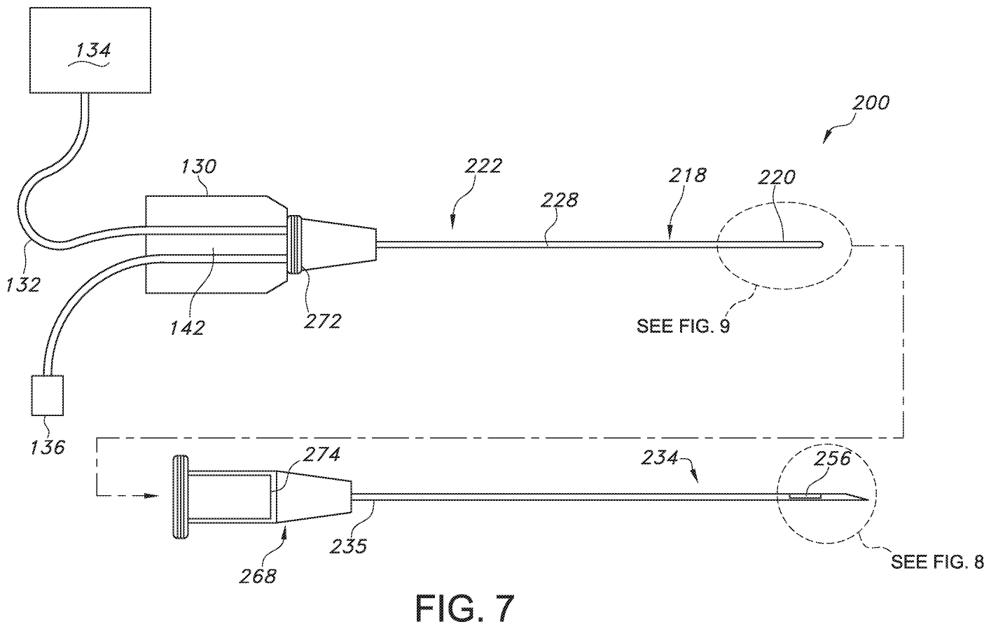

Additionally or alternatively, as shown in FIGS. 7-10, the catheter assembly 200 may be used alone, or placed within an article, such as a needle 234. In such an embodiment, the catheter assembly 200 may be configured to be placed within the lumen of an article, such as a needle, stylet, probe, or guidewire.

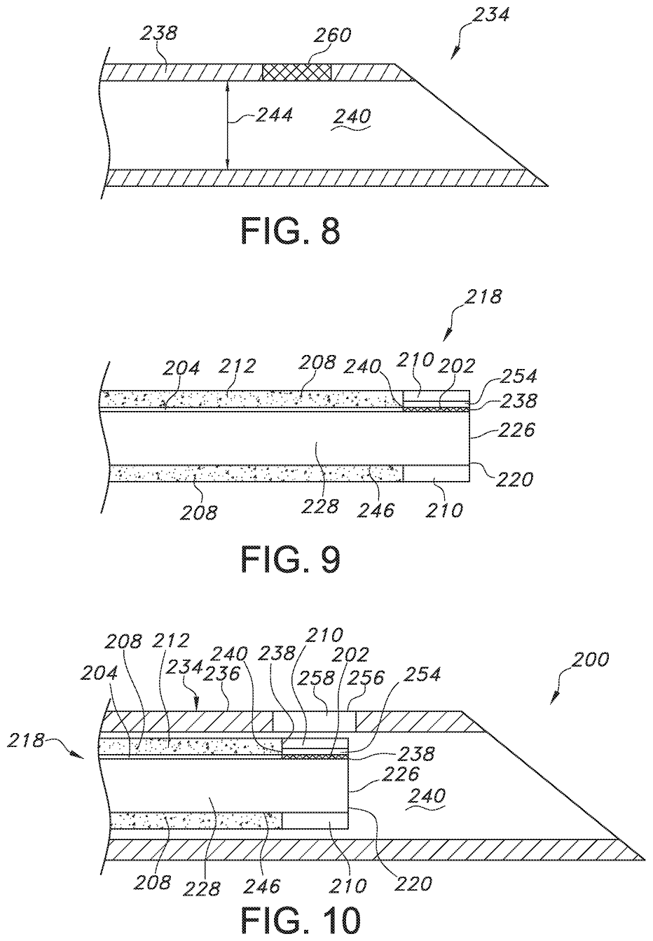

For instance, as shown in FIGS. 7-10, the catheter 218 is designed to be received within the lumen 240 of the needle 234. More specifically, as shown, the catheter 218 also has a proximal end 222 and a distal end 220. Further, as in the embodiments of FIGS. 3-6 above, the ultrasound transducer 202 may be mounted on the catheter 218, e.g. at the distal end 220 thereof. As such, during use, the distal end 220 of the catheter 218 can be inserted into the lumen 240 of the needle 234 that can then together be inserted into a patient. Similar to the embodiments discussed above, and as shown more clearly in FIG. 9, the catheter 218 may have a body 228 having an outer diameter ranging from about 1 millimeters to about 12 millimeters, such as from about 2 millimeters to about 11 millimeters, such as from about 3 millimeters to about 10 millimeters, such as from about 5 millimeters to about 9 millimeters, or any range therebetween, and defined by a distal end 220 and a proximal end 222. Of course, a catheter 218 such as shown in FIGS. 7-10 may also have a smaller or larger outer diameter than as discussed above, and may instead have a outer diameter selected based upon the diameter 244 of the lumen 240 of the needle 234 (see, FIG. 8). A transducer 202 (e.g., an ultrasound transducer) can be disposed on an outer surface 246 of the catheter 218.

Further, as discussed above, the distal end 220 of the catheter 218 can, in one embodiment, have a tip 226 that can be used for the delivery of energy or treatments suitable for creating a nerve block, such as for the treatment of pain, or may instead utilize openings 244 in the distal end 220 for the delivery of energy or treatments. For instance, the tip 226 and/or distal end 220 can include an opening 244 or openings 244 (such as, in FIG. 9, an open end located at tip 226) for delivery of a fluid and/or medicament, such as for delivery of an anesthetic nerve block.

In one embodiment that includes a catheter assembly 200 contained within an article such as a needle 234, the needle 234 further may include at least one window 256 or opening in an outer wall thereof. Thus, as shown, the transducer 202 of the catheter 218 can be aligned with the window(s) 256 when the catheter 218 is inserted into the lumen 240 of the needle 234. In such embodiments, the ultrasound transducer 202 is configured to transmit or divert signals 258 through the window(s) 256. Additionally or alternatively, in such an embodiment, the needle 234 may include a transducer (not shown) in addition to the transducer 202 located on the catheter 218. When used, the transducer on the needle 234 may be generally located at or near the location of the window(s) 256 of FIGS. 7 and 10, or may be located in an alternative position. In such an embodiment, the transducer 260 on the needle 234 may be used for the initial position of the catheter assembly 200, and the transducer 202 located on the catheter 218 may be used to check, or relocate, the catheter assembly 200 after placement of the catheter assembly 200 and removal of the needle 234 from around the catheter 218.

Nonetheless, in an embodiment that includes a catheter assembly configured to be contained within an article such as a needle 234, during use, the distal end of the catheter 218 can be inserted into the lumen 240 of the needle 234 that can then together be inserted into a patient. Additionally or alternatively, the needle 234 may be inserted into the patient, and the catheter 218 may be introduced through the needle 234 after placement of the needle.

As discussed in reference to FIGS. 3-6, the embodiment shown in FIGS. 7-10 may also include a handle 130 that can be coupled to the proximal end 222 of the needle 234 or catheter 218 to assist the medical professional 312 in accurately positioning the catheter 218 within the patient 210. The handle 130 can be connected via an electrical connection 132 to a power supply 134. The power supply 134 can provide energy to the transducer 202 on the catheter 218, and the transducer may be any transducer as discussed above.

Still referring to FIG. 7, the needle 234 may also include a needle hub 268 at its proximal end 235. Thus, in certain embodiments, the handle 130 may be sized to fit within the needle hub 235. In addition, as shown, handle 130, and the needle hub 268 may each be configured with one or more locking features 272, 274 configured to secure the handle 130 to the needle hub 268. More specifically, the locking features 272, 274 may correspond to one or more of ribs, protrusions, fasteners, or similar or combinations thereof. For example, as shown, the protrusion 272 of the handle 230 may be configured to snap fit with the recess 274 of the needle hub 268

As shown, the transducer 202 can have a distal end 238 and a proximal end 240, where one or more transducer wires 204, which can connect the transducer 202 to a power supply and which can be used for transmitting and receiving signals, extends from the proximal end 240 of the transducer 202 towards the proximal end 222 of the catheter 218 in order to connect the transducer 202 to the power supply 234. The one or more transducer wires 204, which can extend along the outer surface 246 of the catheter 218, can be formed from any conductive material and have a size and diameter as discussed above. As discussed above, the ability of the catheter assembly 200 of the present invention to use one or more transducer wires 204 having such a reduced diameter compared to conventional transducer wires where a larger, more cumbersome coaxial cable is required to shield the one or more transducer wires from electrical signal noise is facilitated by the use of a length of tubing 206 surrounding the catheter 218 and the transducer 202, as discussed in more detail below.

As in the embodiments discussed above, the embodiments shown in FIGS. 7-10 may also include a length of tubing 206 that surrounds the catheter 218, the transducer 202, and the one or more transducer wires 204, which can be non-coaxial as discussed above. Specifically, the length of tubing 206 can include a distal portion 210 and a proximal portion 212. The distal portion 210 can extend along the body 228 towards the tip 226 of the catheter 218 such that the tip 226 or some or all of the distal portion 210 of the catheter 218 is exposed and free of the length of tubing 206. However, in this embodiment, such as shown in FIG. 10, the tubing 206 is located between the exterior wall 246 of the catheter, and the interior wall 262 of the needle 234. Therefore, while the tubing 206 may have the same thickness as discussed above, the tubing 206 may also have a thickness selected that enables the catheter 218 to be placed in the lumen 240 of the needle 236. Notwithstanding the thickness of the tubing 206, the tubing 206 may generally have the same properties and be formed as discussed above.

Further, the proximal portion 212 of the length of tubing 206 can include a conductive filler 208 (e.g., conductive particles, powder, flakes, etc.) that helps shield the one or more transducer wires 204 from electrical signal noise that may hinder the effectiveness of the transducer 202 in enhancing the visibility of the distal end 220 of the catheter 218. Meanwhile, the distal portion 210 of the length of tubing 206 which surrounds the transducer 202 is free of the conductive filler 208 so as to not distort the signals that may be transmitted and received by the transducer 202, such as when used the medical imaging system 100 to visualize the distal end 220 of the catheter 218.

As discussed above, the length of tubing 206 can include any suitable material that is utilized in medical tubing, such as polytetrafluoroethylene, polyethylene, polyurethane, silicone, or a combination thereof. Further, the length of tubing 206 can be shrinkable, where the length of tubing can secure the transducer 202 and the one or more transducer wires 204 against the outer surface 246 of the catheter 218 when the length of tubing 206 is shrunk around the body 228 of the catheter 218. In one particular embodiment, the length of tubing 206 can be heat shrinkable.

In addition, any suitable conductive filler 208 can be used, such as a metal, a conductive polymer, carbon, or a combination thereof. For instance, the conductive filler 208 can include nickel, copper, silver, gold, platinum, or a combination thereof. In other embodiments, the conductive filler 208 can include polypyrrole, poly(3,4-ethylenedioxythiophene), polythiophene, polyaniline, poly-p-phenylene-sulphide, polyacetylene, polyisoprene, polybutadiene, or a combination thereof. Moreover, regardless of the particular conductive filler 208 utilized, the conductive filler 208 can be present in the proximal portion 212 of the length of tubing 206 in an amount ranging from about 0.05 wt. % to about 10 wt. %, such as from about 0.1 wt. % to about 7.5 wt. %, such as from about 0.5 wt. % to about 5 wt. % based on the total weight of the proximal portion 212 of the length of tubing 206.

Referring now to FIG. 7, the catheter assembly 200 as described above can also include an injection port 236 coupled to the catheter side handle 230 whereby fluid or medicament can be injected into a proximal end 222 of the catheter 118. In addition, the catheter assembly 200 can also include an article insertion port 242 located at the needle side handle 231 whereby the catheter 218 can be inserted into the needle 234.

As discussed above, although not required, in some embodiments, the catheter assembly 200 can include an impedance matching material 254 disposed between the transducer 202 and the distal portion 210 of the tubing 206. The impedance matching material 254 can eliminate any air between the transducer 202 and the distal portion 210 of the tubing 206, where air could attenuate or otherwise negatively impact the signals being transmitted and received by the transducer 202 via the one or more transducer wires 204. In addition, the impedance matching material 254 can have an acoustic impedance that is similar to the impedance of human tissue (e.g., skin), such as an acoustic impedance that is +/−20%, such as +/−15%, such as +/−10%, such as +/−5% of the impedance of human tissue. By having a similar acoustic impedance as human tissue, the impedance matching layer 254 can reduce the possibility of any reflection of the signals being transmitted and received by the transducer 202 via the one or more transducer wires 204. For instance, in one embodiment, the impedance matching layer can be silicone, polyimide, or any other polymer having an acoustic impedance similar to that of human tissue. In addition, the impedance matching material 254 can be applied to the catheter assembly 200 by disposing, such as by coating, dabbing, painting, brushing, etc., a thin layer of the impedance matching material 254 on a surface of the transducer 202 and the distal end 220 of the catheter 218 around which the tubing 206 will be disposed. Further, the impedance matching material 254 and the tubing 206 can be applied around the catheter 218 in a vacuum chamber to ensure that any air is eliminated between the catheter 218 and the tubing 206.

The present invention has been described both in general and in detail by way of examples. These and other modifications and variations of the present invention may be practiced by those of ordinary skill in the art, without departing from the spirit and scope of the present invention. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed is:

1. A catheter assembly comprising:
a catheter having a catheter body defining a proximal end and a distal end,
a tip located at the distal end of the catheter, the tip being capable of being inserted into a body of a mammal;
a transducer located at the distal end of the catheter proximal to the tip;
one or more transducer wires extending from a proximal end of the transducer towards a proximal end of the catheter; and
a length of tubing surrounding the catheter, the transducer, and the one or more transducer wires, wherein the length of tubing has a proximal portion and a distal portion, wherein the proximal portion surrounds the one or more transducer wires and the distal portion surrounds the transducer.

2. The catheter assembly of claim 1, wherein the transducer is disposed on an outer surface of the catheter.

3. The catheter assembly of claim 2, wherein the length of tubing holds the transducer in place against the outer surface of the catheter.

4. The catheter assembly of claim 1, wherein the length of tubing comprises polytetrafluoroethylene, polyethylene, polyurethane, silicone, or a combination thereof.

5. The catheter assembly of claim 1, wherein the length of tubing is heat shrinkable.

6. The catheter assembly of claim 1, wherein the proximal portion of the tubing includes a conductive filler.

7. The catheter assembly of claim 6, wherein the conductive filler comprises a metal, a conductive polymer, carbon, or a combination thereof.

8. The catheter assembly of claim 7, wherein the conductive filler comprises nickel, copper, silver, gold; platinum, or a combination thereof.

9. The catheter assembly of claim 7, wherein the conductive filler comprises polypyrrole, poly(3,4-ethylenedioxythiophene), polythiophene, polyaniline, poly-p-phenylene-sulphide, polyacetylene, polyisoprene, polybutadiene, or a combination thereof.

10. The catheter assembly of claim 6, wherein the conductive filler is present in the proximal portion of the length of tubing in an amount ranging from about 0.05 wt. % to about 10 wt. % based on the total weight of the proximal portion of the length of tubing.

11. The catheter assembly of claim 1, wherein the one or more transducer wires has a diameter ranging from about 15 micrometers to about 300 micrometers.

12. The catheter assembly of claim 1, wherein a human tissue impedance matching material is disposed between the transducer and the distal portion of the tubing.

13. The catheter assembly of claim 1, wherein the distal end of the catheter includes at least one aperture.

14. The catheter assembly of claim 1, wherein the transducer is electrically connected to an external power supply via the one or more transducer wires.

15. The catheter assembly of claim 1, wherein the transducer transmits and receives signals for detection by an ultrasound imaging system, wherein the transducer enhances the visibility of the distal end of the catheter on an ultrasound image.

16. The catheter assembly of claim 1, further comprising an article configured to be received in the lumen of the catheter.

17. The catheter assembly of claim 16, wherein the article is a needle, stylet, probe, or guidewire.

18. The catheter assembly of claim 1, wherein the catheter assembly is configured to be received in the lumen of an article.

19. The catheter assembly of claim 18, wherein the catheter assembly is disposed within the lumen of an article.

20. The catheter assembly of claim 19, wherein the article is a needle, stylet, probe, or guidewire.
